(19) United States
(12) Patent Application Publication
(10) Pub. No.: US 2005/0037036 A1
(43) Pub. Date: Feb. 17, 2005

(54) Cosmetic and dermatological preparations in the form of o/w-emulsions containing sterols and/or c12-c40 fatty acids

(76) Inventors: Jens Nielsen, Henstedt-Ulzburg (DE); Thomas Raschke, Pinneberg (DE); Heidi Riedel, Hamburg (DE)

Correspondence Address: GREENBLUM & BERNSTEIN, P.L.C., 1950 ROLAND CLARKE PLACE, RESTON, VA, 20191, US

(21) Appl. No.: 10/485,263

(22) PCT Filed: Aug. 6, 2002
(86) PCT No.: PCT/EP02/08743

(30) Foreign Application Priority Data
Aug. 10, 2001 (EP) 101 39 580.9

(51) Int. Cl. A61K 7/00

(52) U.S. Cl. 424/401

(57) ABSTRACT

A cosmetic or dermatological composition which comprises a sterol and/or a C₁₂-C₄₀-fatty acid, an ester of a C₁₂-C₄₀-fatty acid with glycerol and/or a glycol, an ester of a C₁₂-C₄₀-fatty acid and sorbitan, an ethoxylated C₁₂-C₄₀-fatty acid and a C₁₂-C₄₀-fatty alcohol.

The present invention relates to cosmetic and dermatological emulsions, in particular skin-care cosmetic and dermatological emulsions. In one advantageous embodiment, the present invention relates to a use which permits an increase in the stability of fatty-acid-containing preparations, in particular emulsions, preferably of O/N emulsions.

The skin is the largest human organ. Among its many functions (for example for temperature regulation and as a sensory organ) the barrier function, which prevents the skin (and ultimately the entire organism) from drying out, is the most important. At the same time, the skin acts as a protective device against the penetration and absorption of external substances. This barrier function is effected by the epidermis, which, as the outermost layer, forms the actual protective sheath against the environment. Providing about one tenth of the total thickness, it is also the thinnest layer of the skin.

The epidermis is a stratified tissue in which the outer layer, the horny layer (Stratum corneum), is the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipid membrane in the intercellular spaces, which is of complex composition, corresponds to the mortar. This system is essentially a physical barrier to hydrophilic substances, but, because of its narrow and multilayered structure, can equally also be passed by lipophilic substances only with difficulty.

The present invention relates, in a particular embodiment, to cosmetic or pharmaceutical preparations having a reduced feel of stickiness, to processes for their preparation, and to the use of active ingredients for reducing the feel of stickiness of cosmetic preparations.

Apart from their barrier action against external chemical and physical influences, the epidermal lipids also contribute to the holding together of the horny layer and have an effect on the smoothness of the skin. In contrast to the sebaceous gland lipids, which do not form a continuous film on the skin, the epidermal lipids are distributed over the entire horny layer.

The extremely complex interaction of the moisture-binding substances and of the lipids of the upper layers of the skin is very important for the regulation of skin moisture. For this reason, cosmetics generally comprise, in addition to balanced lipid mixtures and water, water-binding substances.

As well as the chemical composition, however, the physical behavior of these substances is also of importance. The development of very biocompatible emulsifiers and surfactants is therefore desirable. Products formulated therewith aid the liquid-crystalline organization of the intercellular lipids of the Stratum corneum, thereby improving the barrier properties of the horny layer. It is particularly advantageous if their molecular constituents consist of substances which are naturally occurring in the epidermis.

Cosmetic skin care primarily means that the natural function of the skin as a barrier against environmental influences (e.g. dirt, chemicals, microorganisms) and against the loss of endogenous substances (e.g. water, natural fats, electrolytes) is strengthened or restored.

If this function is impaired, increased resorption of toxic or allergenic substances or attack by microorganisms may result, leading to toxic or allergic skin reactions.

Another aim of skin care is to compensate for the loss by the skin of lipids and water caused by daily washing. This is particularly important when the natural regeneration ability is insufficient. Furthermore, skincare products should protect against environmental influences, in particular against sun and wind, and delay skin aging.

Medicinal topical compositions generally comprise one or more medicaments in an effective concentration. For the sake of simplicity, in order to distinguish clearly between cosmetic and medicinal use and corresponding products, reference is made to the legal provisions in the Federal Republic of Germany (e.g. Cosmetics Directive, Foods and Drugs Act).

Customary cosmetic forms of application are emulsions. This term generally means a heterogeneous system of two liquids which are immiscible or miscible only to a limited extent with one another, which are usually referred to as phases. One is in the form of droplets (disperse or internal phase), while the other liquid forms a continuous (coherent or internal) phase. Less common forms of application are multiple emulsions, i.e. those which, in the droplets of the dispersed (or discontinuous) phase, comprise for their part droplets of a further dispersed phase, e.g. W/O/W emulsions and O/W/O emulsions.

More recent findings have recently led to a better understanding of cosmetic emulsions which are of relevance in practice. Here, it is assumed that the emulsifier mixtures used in excess form lamellar liquid-crystalline phases or crystalline gel phases. In the gel network theory, stability and physicochemical properties of such emulsions are attributed to the formation of viscoelastic gel networks.

In order to be able to ensure the metastability of emulsions, interface-active substances, i.e. emulsifiers, are usually necessary. The use per se of customary cosmetic emulsifiers is entirely acceptable. Nevertheless, emulsifiers, as ultimately any chemical substance, may in certain cases cause allergic reactions or reactions based on oversensitivity of the user. For example, it is known that in some particularly sensitive people, certain light dermatoses are triggered by certain emulsifiers and simultaneous action of sunlight.

It is possible to prepare emulsifier-free preparations which, for example, have, in an aqueous phase, dispersed oil droplets, similar to an O/W emulsion. A prerequisite for this may be that the continuous aqueous phase has a gel framework which stabilizes the dispersed phase, and other conditions besides. Such systems are sometimes called hydrodispersions or oleodispersions depending on which is the disperse phase and which is the continuous phase.

For cosmetics technology, it is neither necessary nor possible to dispense with emulsifiers altogether, especially since there is a certain choice of particularly mild emulsifiers. However, the prior art lacks a satisfactorily broad range of such emulsifiers which would then also significantly broaden the application spectrum of correspondingly mild cosmetic preparations which are tolerated by the skin.

It was thus an object of the present invention to provide cosmetic and/or dermatological preparations with excellent skincare properties.

It was also an object of the present invention to provide preparations which clearly improve the condition of skin, in particular reduce the roughness of skin.

Although it is known to reduce a feeling of stickiness or else a feeling of greasiness by adding certain substances, for example some selected powder raw materials, in particular talc, apart from the fact that this is only rarely completely possible, such an addition also changes the viscosity of the product in question and reduces the stability.

The object was therefore to remedy all of these the disadvantages of the prior art. In particular, the intention was to provide products having reduced stickiness or greasiness.

Products in the field of care cosmetics, decorative cosmetics and pharmacological technology should likewise be freed from the described disadvantages of the prior art.

Furthermore, it was an object of the invention to develop cosmetic bases for cosmetic preparations which are characterized by good skin compatibility.

Known cosmetic preparations are so-called stearate emulsions, i.e. those in which stearic acid and/or palmitic acid or alkali metal salts of stearic acid and/or of palmitic acid are effective as emulsifier. These preparations can advantageously be in the form of O/W emulsions and are characterized by a good feel on the skin. A disadvantage, however, is that fatty acids in a pH range from 3.5-8.0 have a tendency toward crystallization (in particular in a pH range below 7.0), as a result of which the pleasant feel on the skin and the external appearance of a corresponding preparation are severely impaired.

The person skilled in the art is of course aware of a large number of ways to formulate stable W/O preparations for cosmetic or dermatological use, for example in the form of creams and ointments which can be spread in the range from room temperature to skin temperature, or as lotions and milks, which are more likely flowable in this temperature range. However, there are only a few formulations in the prior art which are of sufficiently low-viscosity that they would, for example, be sprayable.

In addition, low-viscosity preparations of the prior art frequently have the disadvantage that they are unstable, and are limited to a narrow field of application or a limited choice of feed materials. Low-viscosity products in which, for example, strongly polar oils—such as the plant oils otherwise frequently used in commercially available products—are sufficiently stabilized, are therefore currently not on the market.

The term “viscosity” means the property of a liquid to resist the mutual laminar displacement of two neighboring layers (internal friction). This so-called dynamic viscosity is nowadays defined according to η=τ/D as the ratio of shear stress to the velocity gradient perpendicular to the direction of flow. For Newtonian liquids, η is a material constant having the Si unit Pascal second (Pa·s) at a given temperature.

The quotient ν=θ/ρ from the dynamic viscosity η and the density ρ of the liquid is referred to as the kinematic viscosity ν and is given in the Si unit m²/s.

Fluidity (φ) is the inverse of viscosity (φ=1/η). In the case of ointments and the like, the use value is inter alia codetermined by the so-called tack. The tack of an ointment or ointment base or the like means its property to draw threads of varying lengths when a small sample is removed; accordingly, a distinction is made between short- and long-stretch substances.

While the graphical representation of the flow behavior of Newtonian liquids at a given temperature produces a straight line, in the case of so-called non-Newtonian liquids considerable deviations often arise, depending on the velocity gradient D (shear rate γ) or the shear stress τ. In these cases, the so-called apparent viscosity can be determined which, although it does not obey the Newtonian equation, can be used to determine the true viscosity values by graphical methods.

Falling-body viscometry is suitable only for investigating Newtonian liquids and gases. It is based on Stokes's law, according to which, for the falling of a sphere through a liquid which flows around it, the dynamic viscosity η can be determined from

$$η = \frac{2 {r}^{2} ( {ρ}_{K} - {ρ}_{FI} ) \cdot g}{9 \cdot v}$$

where

r=radius of the sphere, ν=fall velocity, ρ_{κ}=density of the sphere, ρ_{FI}=density of the liquid and g=acceleration of the fall.

O/W emulsions with a low viscosity which have a storage stability as is required for marketable products can only be formulated in accordance with the prior art in a very involved process.

Accordingly, the supply of formulations of this type is extremely low. Nevertheless, such formulations could offer the consumer as yet unknown cosmetic effects.

It was also an object of the present invention to provide products with the broadest possible application diversity. For example, the aim was to provide bases for preparation forms such as cleansing emulsions, face and bodycare preparations, but also extremely medicinal-pharmaceutical presentation forms, for example preparations against acne and other skin phenomena.

Surprisingly, it has been found, and herein lies the achievement of these objects, that cosmetic or dermatological preparations in the form of the O/W emulsion comprising
(I) 1 to 5% by weight, based on the total weight of the preparations, of one or more substances chosen from the group of sterols, branched or unbranched, saturated or unsaturated C₁₂-C₄₀-fatty acids,
(II) 0.1 to 1.5% by weight, based on the total weight of the preparations, of one or more mono-, sesqui-, di-, triesters of saturated or unsaturated, straight-chain or branched-chain fatty acids with a chain length of C₁₄-C₄₀ of the glycerol and/or of the propylene glycol and/or of the glycol,
(III) 0.1 to 1.5% by weight, based on the total weight of the preparation, of one or more mono-, sesqui-, di-, triesters of saturated or unsaturated, straight-chain or branched-chain fatty acids with a chain length of C₁₄-C₄₀ of the sorbitan and optionally with a degree of polyethoxylation of 0-100,
(IV) 0.1 to 1.5% by weight, based on the total weight of the preparations, of one or more ethoxylated fatty acid esters with fatty acids of chain length C₁₂-C₄₀ and a degree of ethoxylation up to 100, preferably from 5-100,
(V) 0.5-7% by weight, based on the total weight of the preparations, of one or more fatty alcohols chosen from the group of branched and unbranched, saturated and unsaturated alkyl alcohols having 12 to 40 carbon atoms
(VI) where the ratio of (II):(III):(IV) is preferably chosen as a:b:c, where a, b and c, independently of one another, are rational numbers from 1 to 5, preferably from 1 to 3,
(VII) and the ratio of (II)+(III)+(IV) to (V) is preferably in the range 5:1 to 1:5,
(VIII) where the sum of (I), (II), (III), (IV) and (V) is at most 25% by weight,
(IX) and where the preparations are advantageously present in a pH range 3.5-8.0, preferably at pH values of 4.5-6.5,
   overcome the disadvantages of the prior art.

It had therefore not been foreseen by the person skilled in the art that the preparations according to the invention
have better effectiveness as moisture-donating preparations,
better promote skin smoothing,
are characterized by better care action,
better serve as vehicles for cosmetic and medicinal-dermatological active ingredients
have higher stability against crystallization of the fatty acids used and
would be characterized by better biocompatibility
would be characterized by a better feel on the skin and by higher cosmetic elegance
would be characterized over a broad cosmetic variability and would be able to be formulated over broad consistency and viscosity ranges from 400 mPas to >20 000 mPas
   than the preparations of the prior art.

The preparations according to the invention can be formulated to be sprayable, flowable or else cream-like, have very good cosmetic properties, in particular with regard to stickiness, and have a very good skin compatibility and skincare effect.

Sterols are steroids which only carry a hydroxyl group in the 3 position, but otherwise no functional group, and are thus formally alcohols. In addition, the sterols, which contain 27 to 30 carbon atoms, generally have a double bond in the 5/6 position, more rarely also/or in 7/8, 8/9 and other positions (e.g. 22/23).

The sterols are widespread in nature as lipids—mostly in the form of esters (formally called sterides). The sterols which occur in the animal kingdom are called zoosterols. The most important representative is cholesterol. Further zoosterols are found in wool fat (lanosterol, dihydrolanosterol), in the silk worm, in sponges (spongosterol), starfish, sea urchins, oysters etc.

Cholesterol is characterized by the following structure:

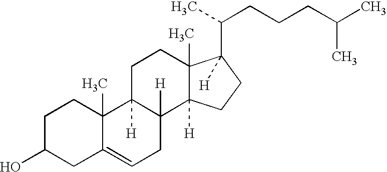

Lanosterol is characterized by the following structure:

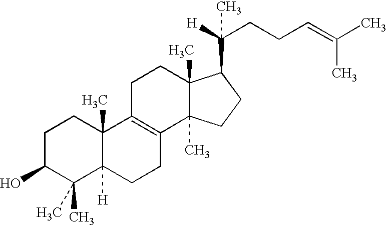

Dihydrolanosterol is characterized by the following structure:

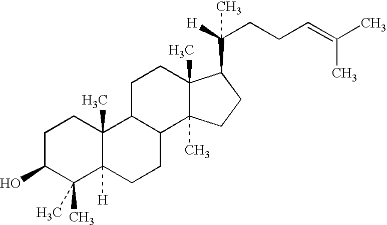

The plant sterols are called phytosterols. Their most important representatives are ergosterol, stigmasterol and sitosterol. Some are used in cosmetic products. Sometimes, the sterols from fungi and yeasts are separated as mycosterols (e.g. ergosterol, fungisterol, stellasterol and zymosterol) from the group of phytosterols.

Examples of phytosterols are:

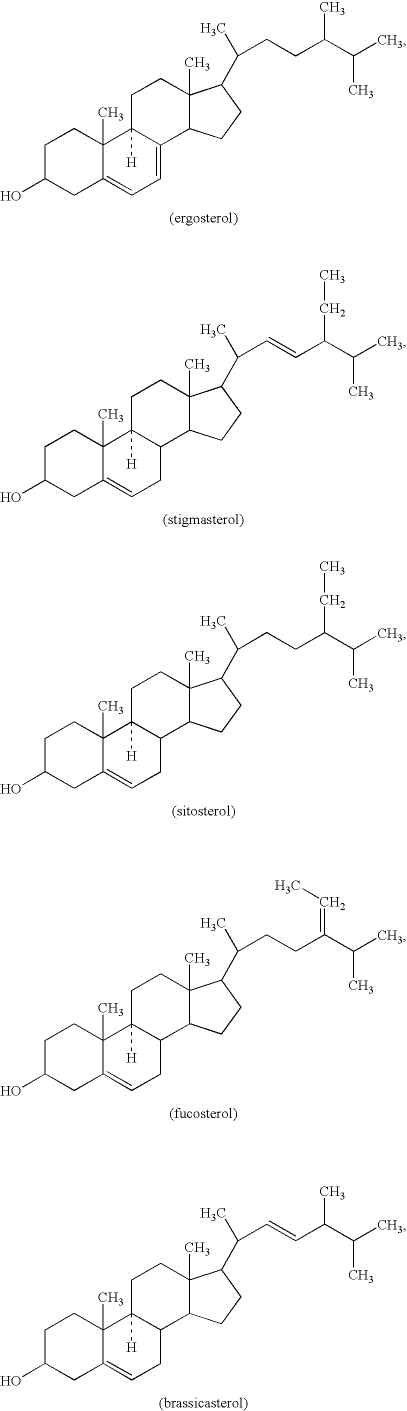

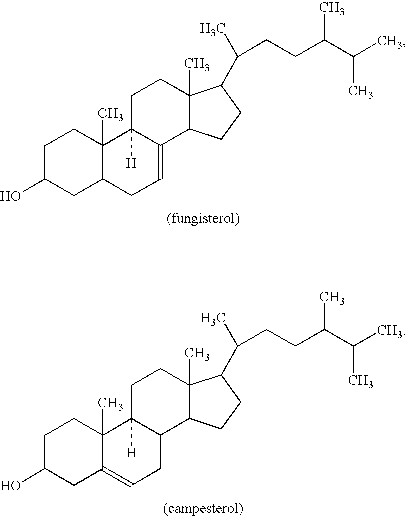

All of the phytosterols to be used according to the invention have a greater or lesser number of optical isomers, which will not be listed individually here, but which have proven advantageous provided their cosmetic acceptability is not an issue.

Preferred sterols are cholesterol and lanosterol.

Preferred fatty acids are steric acid and/or palmitic acid (stearin) and lignoceric acid.

Advantageous embodiments of the present invention relate to cosmetic and dermatological preparations comprising
(II) 0.1 to 1.5% by weight, based on the total weight of the preparations, of one or more glycol or glycerol esters chosen from the group of mono-, sesqui-, di-, triesters of glycols (=vic-diols or 1,2-diols) or of glycerol (1,2,3-trihydroxpropanes) with fatty acids of chain length C₁₂-C₄₀ (even-numbered, saturated, unsaturated and branched). Examples: glycerol monostearate, glycerol distearate, propylene glycol monostearate, glycerol isostearate, glycerol lanolate, glycerol myristate, glycerol laurate, glycerol oleate, glycerol stearate citrate.

Advantageous embodiments of the present invention relate to cosmetic and dermatological preparations comprising
(III) 0.1 to 1.5% by weight of one or more sorbitan esters chosen from the group of mono-, sesqui-, di- and triesters of sorbitans (=monohydrosorbites or 1,5-anhydro-D-sorbitol) with fatty acids of chain length C₁₂-C₄₀ (even-numbered, saturated, unsaturated and branched), and optionally a degree of ethoxylation from 10-100). Examples: sorbitan stearate, sorbitan distearate, sorbitan isostearate, sorbitan oleate, PEG-40 sorbitan peroleate, PEG-40 sorbitan perisostearate, sorbitan sesquioleate.

Advantageous embodiments of the present invention further relate to cosmetic and dermatological preparations comprising
(III) 0.1 to 1.5% by weight, based on the total weight of the preparations, of one or more ethoxylated fatty acid esters with fatty acids of chain length C₁₂-C₄₀ and a degree of ethoxylation of 5-100, in particular chosen from the group consisting of PEG-20 stearate, PEG-30 stearate, PEG-40 stearate, PEG-100 stearate

Advantageous embodiments of the present invention further relate to cosmetic and dermatological preparations comprising
(V) 0.5-7% by weight, based on the total weight of the preparations, of one or more fatty alcohols, chosen from the group consisting of myristyl alcohol, cetyl alcohol, isocetyl alcohol, cetylstearyl alcohol, stearyl alcohol, isostearyl alcohol, behenyl alcohol and mixtures thereof.

The total amount of branched and unbranched alkyl alcohols having 12 to 40 carbon atoms in the finished cosmetic or dermatological preparations is advantageously chosen from the range 0.1-7.0% by weight, preferably 0.5-5.0% by weight, based on the total weight of the preparations.

Wool wax alcohols (CAS No. 8027-33-6) represent the unhydrolyzable alcohol fraction of wool wax which is obtained following the hydrolysis of wool wax. They consist of about 25.2% of cholesterol, of 2.7% of lanosterol, of 2.2% of dihydrolanosterol and of about 29.5% of aliphatic monohydric C₁₆-C₃₂-alcohols. Wool wax alcohols are therefore used for the preparation of ointment bases from which W/O emulsions are mostly prepared.

Advantageous embodiments of the present invention thus relate to preparations according to
(V) where the from the group of branched and unbranched, saturated and unsaturated alkyl alcohols having 12 to 40 carbon atoms are chosen from the group of wool wax alcohols,
(I) where these wool wax alcohols also comprise one or more sterols, for example cholesterol, lanosterol, dihydrolanosterol, as well as the aliphatic alcohols.

Advantageous embodiments of the present invention relate, for example, to cosmetic and dermatological preparations in the form of O/W emulsions comprising 3% of liquid lipids (preferably chosen from (a) Guerbet alcohols, (b) saturated triglycerides and (c) ethers of medium-chain fatty alcohols, (d) nonpolar lipids, (e) silicone oils, (f) dialkyl carbonates or mixtures thereof.

For the purposes of the present disclosure, the expression “lipids” is sometimes used as the generic term for fats, oils, waxes and the like, as is entirely familiar to the person skilled in the art. The terms “oil phase” and “lipid phase” are also used synonymously.

Oils and fats differ from one another, inter alia, in their polarity, which is difficult to define. It has already been proposed to adopt the interfacial tension toward water as a measure of the polarity index of an oil or of an oil phase. This means that the lower the interfacial tension between the oil phase and water, the greater the polarity of the oil phase in question. According to the invention, the interfacial tension is regarded as one possible measure of the polarity of a given oil component.

The interfacial tension is the force which acts on an imaginary line of one meter in length in the interface between two phases. The physical unit for this interfacial tension is conventionally calculated from the force/length relationship and is usually expressed in mN/m (millinewtons divided by meters). It has a positive sign if it endeavors to reduce the interface. In the converse case it has a negative sign. For the purposes of the present invention, lipids are regarded as being polar if their interfacial tension toward water is less than 30 mN/m.

Polar oils are, for example, those from the group of lecithins and of fatty acid triglycerides, namely the triglycerol esters of saturated and/or unsaturated, branched and/or unbranched alkanecarboxylic acids with a chain length from 8 to 24, in particular 12 to 18, carbon atoms. The fatty acid triglycerides can, for example, advantageously be chosen from the group of synthetic, semisynthetic and natural oils, such as e.g. olive oil, sunflower oil, soybean oil, groundnut oil, rapeseed oil, almond oil, palm oil, coconut oil, castor oil, wheatgerm oil, grapeseed oil, thistle oil, evening primrose oil, macadamia nut oil and the like.

Further polar oil components can be chosen from the group of esters of saturated and/or unsaturated, branched and/or unbranched alkanecarboxylic acids having a chain length of from 3 to 30 carbon atoms and saturated and/or unsaturated, branched and/or unbranched alcohols with a chain length of from 3 to 30 carbon atoms, and from the group of esters of aromatic carboxylic acids and saturated and/or unsaturated, branched and/or unbranched alcohols of chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate and 2-octyldodecyl palmitate, oleyl oleate, oleyl erucate, erucyl oleate, erucyl erucate, and synthetic, semisynthetic and natural mixtures of such esters, such as, for example, jojoba oil.

In addition, the oil phase can advantageously be chosen from the group of dialkyl ethers, the group of saturated or unsaturated, branched or unbranched alcohols. It is particularly advantageous if the oil phase of the W/O emulsions according to the invention has a content of C₁₂₋₁₅-alkyl benzoate or consists entirely of the latter.

In addition, the oil phase can advantageously be chosen from the group of Guerbet alcohols. Guerbet alcohols are named after Marcel Guerbet who described their preparation for the first time. They are formed according to the reaction equation 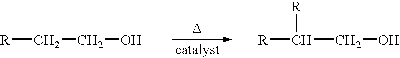
by oxidation of an alcohol to an aldehyde, by aldol condensation of the aldehyde, elimination of water from the aldol and hydrogenation of the allyl aldehyde. Guerbet alcohols are liquid even at low temperatures and bring about virtually no skin irritations. They can be used advantageously as fatting, superfatting and also refatting constituents in skincare and haircare compositions.

The use of Guerbet alcohols in cosmetics is known per se. Such species are then in most cases characterized by the structure

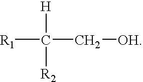

Here, R₁ and R₂ are usually unbranched alkyl radicals.

According to the invention, the Guerbet alcohol(s) is/are advantageously chosen from the group in which
R₁=propyl, butyl, pentyl, hexyl, heptyl or octyl and
R₂=hexyl, heptyl, octyl, nonyl, decyl, undecyl, dodecyl, tridecyl or tetradecyl.

Guerbet alcohols which are preferred according to the invention are 2-butyloctanol—it has the chemical structure 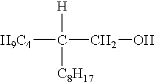
and is available, for example, under the trade name Isofol® 12 from Condea Chemie GmbH—and 2-hexyldecanol—it has the chemical structure 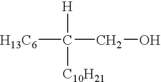
and is available, for example, under the trade name Isofol® 16 from Condea Chemie GmbH.

Mixtures of Guerbet alcohols according to the invention can also advantageously be used according to the invention. Mixtures of 2-butyloctanol and 2-hexyldecanol are available, for example, under the trade name Isofol® 14 from Condea Chemie GmbH.

The total amount of Guerbet alcohols in the finished cosmetic or dermatological preparations is advantageously chosen from the range up to 25.0% by weight, preferably 0.5-15.0% by weight, based on the total weight of the preparations.

Any mixtures of such oil and wax components can also be used advantageously for the purposes of the present invention. It may also be advantageous to use waxes, for example cetyl palmitate, as the sole lipid component of the oil phase.

Nonpolar oils are, for example, those which are chosen from the group of branched and unbranched hydrocarbons and hydrocarbon waxes, in particular vaseline (petrolatum), paraffin oil, squalane and squalene, polyolefins and hydrogenated polyisobutenes. Among the polyolefins, polydecenes are the preferred substances. Table 1 below lists lipids which are advantageous according to the invention as individual substances and also as mixtures with one another. The corresponding interfacial tensions toward water are given in the last column. It is, however, also advantageous to use mixtures of greater or lesser polar and the like.

TABLE 1

| Trade name | INCI name | (mN/m) |
|---|---|---|
| Isofol ® 14 T | Butyl Decanol + Hexyl | 27.6 |
| | Decanol + Hexyl Octanol + | |
| | Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Miglyol ® 812 | Caprylic/Capric Triglyceride | 21.3 |
| Cegesoft ® C24 | Octyl Palmitate | 23.1 |
| Isopropyl Stearate | Isopropyl Stearate | 21.9 |
| Estol ® 1540 EHC | Octyl Octanoate | 30.0 |
| Finsolv ® TN | C₁₂₋₁₅ Alkyl Benzoate | 21.8 |
| Cetiol ® SN | Cetearyl Isonoanoate | 28.6 |
| Dermofeel ® BGC | Butylene Glycol | 21.5 |
| | Caprylate/Caprate | |
| Trivent ® OCG | Tricaprylin | 20.2 |
| MOD | Octyldodeceyl Myristate | 22.1 |
| Cosmacol ® ETI | Di-C₁₂₋₁₃ Alkyl Tartrate | 29.4 |
| Miglyol ® 829 | Caprylic/Capric Diglyceryl | 29.5 |
| | Succinate | |
| Prisorine ® 2036 | Octyl Isostearate | 29.7 |
| Tegosoft ® SH | Stearyl Heptanoate | 28.7 |
| Abil ® Wax 9840 | Cetyl Dimethicone | 25.1 |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

Basic constituents of the preparations according to the invention which may be used are:
water or aqueous solutions
aqueous ethanolic solutions
natural oils and/or chemically modified natural oils and/or synthetic oils;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

The oil phase of the emulsions for the purposes of the present invention consists, according to the invention, preferably predominantly of components of the type listed under point (4), although it is possible without great detriment, to choose up to 50% by weight, preferably up to 40% by weight, of the total weight of the oil components from the group of other oil components. These can then advantageously be chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, from the group of esters of aromatic cargboxylic acids and saturated and/or unsaturated, branched and/or unbranched alcohols of chain length of from 3 to 30 carbon atoms. Such ester oils can then advantageously be chosen from the group consisting of isopropyl myristate, isopropyl palmitate, isorpopyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, the dialkyl ethers, the group of saturated or unsaturated, branched or unbranched alcohols, and the fatty acid triglycerides, namely the triglycerol esters of saturated and/or unsaturated, branched and/or unbranched alkanecarboxylic acid of chain length of from 8 to 24, in particular 12-18, carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like, provided the conditions required in the main claim are observed.

Fatty and/or wax components which are to be used advantageously according to the invention can be chosen from the group of plant waxes, animal waxes, mineral waxes and petrochemical waxes. Examples which are favorable according to the invention are candelilla wax, carnauba wax, Japan wax, esparto grass wax, cork wax, guaruma wax, ricegerm oil wax, sugarcane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes, provided the conditions required in the main claim are observed.

Further advantageous fatty and/or wax components are chemically modified waxes and synthetic waxes, such as, for example, those available under the trade names Syncrowax HRC (glyceryl tribehenate), Syncrowax HGLC (C₁₆₋₃₆-fatty acid triglyceride) and Syncrowax AW 1C(C₁₈₋₃₆-fatty acid) from CRODA GmbH, and montan ester waxes, sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated plant oils (for example hydrogenated castor oil and/or hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Also advantageous are certain organosilicon compounds which have similar physical properties to the specified fatty and/or wax components, such as, for example, stearoxytrimethylsilane provided the conditions required in the main claim are observed.

According to the invention, the fatty and/or wax components can be present either individually or in the mixture.

Any desired mixtures of such oil and wax components can also be used advantageously for the purposes of the present invention.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether provided the conditions required in the main claim are observed.

Particularly advantageous mixtures are those of octyldodecanol, caprylic/capric triglyceride, dicaprylyl ether or mixtures of C₁₂₋₁₅-alky benzoate and 2-ethylhexyl isostearate, mixtures of C₁₂₋₁₅-alky benzoate and isotridecyl isononanoate, and mixtures of C₁₂₋₁₅-alky benzoate, 2-ethylhexyl isostearate and isotridecyl isononanoate provided the conditions required in the main claim are observed.

Of the hydrocarbons, paraffin oil, cycloparaffin, squalane, squalene, hydrogenated polyisobutene and polydecene are to be used advantageously for the purposes of the present invention provided the conditions required in the main claim are observed.

O/W emulsions according to the invention can advantageously be prepared using customary O/W emulsifiers, if desired with the aid of W/O emulsifiers or other coemulsifiers.

If desired, O/W emulsions corresponding to the present invention further comprise one or more emulsifiers, if desired advantageously chosen from the group of the following substances, which generally act as W/O emulsifiers:
lecithin, lanolin, microcrystalline wax (Cera microcristallina) in a mixture with paraffin oil (Paraffinum liquidum), ozokerite, hydrogenated castor oil, polyglyceryl-3 oleate, wool wax acid mixtures, wool wax alcohol mixtures, pentaerythrityl isostearate, polyglyceryl-3 diisostearate, beeswax (Cera alba) and stearic acid, sodium dihydroxycetylphosphate in a mixture with isopropyl hydroxycetyl ether, methylglucose dioleate, methylglucose dioleate in a mixture with hydroxystearate and beeswax, mineral oil in a mixture with petrolatum and ozokerite and glyceryl oleate and lanolin alcohol, petrolatum in a mixture with ozokerite and hydrogenated castor oil and glyceryl isostearate and polyglyceryl-3 oleate, PEG-7-hydrogenated castor oil, ozokerite and hydrogenated castor oil, polyglyceryl-4 isostearate, polyglyceryl-4 isostearate in a mixture with cetyidimethicone copolyol and hexyl laurate, laurylmethicone copolyol, cetyidimethicone copolyol, acrylate/C₁₀₋₃₀-alkyl acrylate crosspolymer, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoyl polyglyceryl-3 diisostearate, polyglyceryl-2 dipolyhydroxystearate, polyglyceryl-3 dipolyhydroxystearate, polyglyceryl-4 dipolyhydroxystearate, polyglyceryl-3 dioleate.

If desired, O/W emulsions according to the present invention comprise one or more emulsifiers, particularly advantageously chosen from the group of the following substances, which generally act as O/W emulsifiers:
glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, sodium cetylstearyl sulfate, lecithin trilaureth-4 phosphate, laureth-4 phosphate, stearic acid, propylene glycol stearate SE, PEG-25 hydrogenated castor oil, PEG-54 hydrogenated castor oil, PEG-6 caprylic/capric glycerides, glyceryl oleate in a mixture with propylene glycol, ceteth-2, ceteth-20, polysorbate 60, glyceryl stearate in a mixture wih PEG-100 stearate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, methylglucose sesquistearate, steareth-10, PEG-20 stearate, steareth-2 in a mixture with PEG-8 distearate, steareth-21, steareth-20, isosteareth-20, PEG-45/dodecyl glycol copolymer, methoxy-PEG-22/dodecyl glycol copolymer, PEG-20 glyceryl stearate, PEG-20 glyceryl stearate, PEG-8 beeswax, polyglyceryl-2 laurate, isostearyl diglyceryl succinate, stearamidopropyl PG dimonium chloride phosphate, glyceryl stearate SE, ceteth-20, triethyl citrate, PEG-20 methylglucose sesquistearate, ceteareth-12, glyceryl stearate citrate, cetyl phosphate, triceteareth-4 phosphate, trilaureth-4 phosphate, polyglyceryl methylglucose distearate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, cetylstearyl alcohol and cetyl palmitate, cetylstearyl alcohol in a mixture with PEG-20 stearate, PEG-30 stearate, PEG-40 stearate, PEG-100 stearate.

For the purposes of the present invention, emulsions according to the invention, e.g. in the form of a skin protection cream, a skin lotion, a cosmetic milk, for example in the form of a sunscreen cream or a sunscreen milk, are advantageous and comprise, for example, fats, oils, waxes and/or other fatty bodies, and also water and one or more emulsifiers, as are customarily used for such a type of emulsion.

Just as emulsions of liquid and solid consistency can be used as cosmetic cleansing lotions or cleansing creams, the preparations according to the invention can also represent spray-able cleansing preparations (“cleansing sprays”), which are used, for example, for removing make-up or as mild washing lotion—where appropriate also for bad skin. Such cleansing preparations can advantageously also be applied as so-called rinse-off preparations, which are rinsed off following application to the skin.

The person skilled in the art is of course aware that high-quality cosmetic compositions are in most cases inconceivable without the customary auxiliaries and additives. These include, for example, bodying agents, fillers, perfume, dyes, emulsifiers, additional active ingredients such as vitamins or proteins, light protection agents, stabilizers, insect repellents, alcohol, water, salts, antimicrobial, proteolytic or keratolytic substances, etc.

Corresponding requirements apply mutatis mutandis to the formulation of medicinal preparations.

For the purposes of the present invention, medicinal topical compositions generally comprise one or more medicaments in an effective concentration. For the sake of simplicity, in order to distinguish clearly between cosmetic and medicinal use and corresponding products, reference is made to the legal provisions in the Federal Republic of Germany (for example Cosmetics Directive, Foods and Drugs Act).

Accordingly, for the purposes of the present invention, cosmetic or topical dermatological compositions can, depending on their composition, be used for example as skin protection cream, cleansing milk, sunscreen lotion, nourishing cream, day or night cream, etc. It is in some cases possible and advantageous to use the compositions according to the invention as a base for pharmaceutical formulations.

It is likewise advantageous to make use of the properties according to the invention in the form of decorative cosmetics (make-up formulations).

Those cosmetic and dermatological preparations which are in the form of a sunscreen are also favorable. In addition to the active ingredient used according to the invention, these also preferably comprise in addition at least one UVA filter substance and/or at least one UVB filter substance and/or at least one inorganic pigment.

However, it is also advantageous for the purposes of the present invention to provide cosmetic and demmatological preparations whose main purpose is not protection against sunlight, but which nevertheless contain a content of anti-UV substances. Thus, for example, UV-A and UV-B filter substances are usually incorporated into day creams.

Preparations according to the invention can advantageously comprise substances which absorb UV radiation in the UVB region, the total amount of filter substances being, for example, from 0.1% by weight to 30% by weight, preferably from 0.5 to 10% by weight, in particular from 1 to 6% by weight, based on the total weight of the preparations.

The UVB filters can be oil-soluble or water-soluble. Examples of oil-soluble substances which may be mentioned are:
3-benzylidenecamphor and derivatives thereof, e.g. 3-(4-methylbenzylidene)camphor,
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate;
2,4,6-trianilino(p-carbo-2′-ethyl-1′-hexyloxy)-1,3,5-triazine.

Advantageous water-soluble substances are:
2-phenylbenzimidazole-5-sulfonic acid and salts thereof, for example sodium, potassium or triethanolammonium salts,
sulfonic acid derivatives of benzophenones, preferably 2-hydroxy-4-methoxy-benzophenone-5-sulfonic acid and its salts;
sulfonic acid derivatives of 3-benzylidenecamphor, such as, for example, 4-(2-oxo-3-bomylidenemethyl)benzenesulfonic acid, 2-methyl-5-(2-oxo-3-bornylidenemethyl)sulfonic acid and its salts.

The list of specified UVB filters which can be used according to the invention is of course not intended to be limiting.

It can also be advantageous to use UVA filters that are usually present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations which comprise these combinations are also provided by the invention. It is possible to use the same amounts of UVA filter substances which were specified for UVB filter substances.

For the purposes of the present invention, cosmetic and/or dermatological preparations can also comprise inorganic pigments which are usually used in cosmetics for protecting the skin against UV radiation. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the quantities specified for the above combinations.

The cosmetic and dermatological preparations according to the invention can comprise cosmetic active ingredients, auxiliaries and/or additives as are usually used in such preparations, for example antioxidants, preservatives, bactericides, perfumes, antifoams, dyes, pigments which have a coloring effect, thickeners, surfactants, emulsifiers, emollients, moisturizers and/or humectants, fats, oils, waxes or other usual constituents of a cosmetic or dermatological formulation, such as alcohols, polyols, polymers, foam stabilizers, electrolytes, organic solvents or silicone derivatives.

For the purposes of the present invention, it is advantageous to add other anti-irritant or anti-inflammatory active ingredients to the preparations, in particular batyl alcohol (α-octadecyl glyceryl ether), selachyl alcohol (α-octadecenyl glyceryl ether), chimyl alcohol (α-hexadecyl glyceryl ether), bisabolol and/or panthenol.

It is likewise advantageous to add conventional antioxidants to the preparations for the purposes of the present invention. According to the invention, favorable antioxidants can be any antioxidants which are suitable or customary for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, ferulic acid, furfurylideneglucitol, carnosine, butylhydroxytoluene, butylhydroxyanisole, nordihydroguaiacic resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are used as the antioxidant(s), it is advantageous to choose their respective concentrations from the range 0.001-10% by weight, based on the total weight of the formulation.

Preparations according to the present invention can also be used as bases for cosmetic or dermatological deodorants or antiperspirants. All active ingredients which are common for deodorants or antiperspirants can be used advantageously, for example odor maskers such as the customary perfume constituents, odor absorbers, for example the phyllosilicates described in laid-open patent specification DE patent 40 09 347, and of these, in particular montmorillonite, kaolinite, ilite, beidellite, nontronite, saponite, hectorite, bentonite, smectite, and also, for example, zinc salts of ricinoleic acid.

Antibacterial agents are likewise suitable for incorporation into the preparations according to the invention. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichlorocarbanilide, quaternary ammonium compounds, oil of cloves, mint oil, oil of thyme, triethyl citrate, farnesol (3,7,11-trimethyl-2,6,10-dodecatrien-1-ol) and the active ingredients or active ingredient combinations described in laid-open patent specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate can also be used advantageously.

The amount of such active ingredients (one or more compounds) in the preparations according to the invention is preferably from 0.001 to 30% by weight, particularly preferably 0.05-20% by weight, in particular 1-10% by weight, based on the total weight of the preparation.

For the purposes of the present invention, the water phase of the cosmetic preparations can also have gel character and, in addition to an effective content of the substances used according to the invention and the solvents used customarily therefor, preferably water, also comprises other organic thickeners, e.g. gum arabic, xanthan gum, sodium alginate, starch and starch derivatives (e.g. distarch phosphate), cellulose, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates such as, for example, organically modified and also unmodified hectorites, bentonites or the like, or a mixture of polyethylene glycol and polyethylene glycol stearate or distearate. The thickener is present in the gel, for example in an amount between 0.1 and 30% by weight, preferably between 0.5 and 15% by weight.

It can also be advantageous to add interface- or surface-active agents to preparations according to the invention, for example cationic emulsifiers such as, in particular, quaternary surfactants.

Quaternary surfactants contain at least one nitrogen atom which is covalently bonded to 4 alkyl or aryl groups. Irrespective of the pH, this leads to a positive charge. Alkylbetaine, alkylamidopropylbetaine and alkylamidopropylhydroxysulfaine are advantageous. The cationic surfactants used according to the invention can also preferably be chosen from the group of quaternary ammonium compounds, in particular benzyltrialkylammonium chlorides or bromides, such as, for example, benzyldimethylstearylammonium chloride, and also alkyltrialkylammonium salts, for example for example cetyltrimethylammonium chloride or bromide, alkyldimethylhydroxyethylammonium chlorides or bromides, dialkyldimethyl-ammonium chlorides or bromides, alkylamidoethyltrimethylammonium ether sulfates, alkylpyridinium salts, for example lauryl- or cetylpyrimidinium chloride, imidazoline derivates and compounds having a cationic character such as amine oxides, for example alkyldimethylamine oxides or alkylaminoethyldimethylamine oxide. In particular, cetyltrime-thylammonium salts can be used advantageously.

It is also advantageous to use cationic polymers (e.g. Jaguar® C 162 [hydroxypropyl guar hydroxypropyltrimonium chloride] or modified magnesium aluminum silicates (e.g. quaternium-18 hectorite, which is obtainable, for example, under the trade name Bentone® 38 from Rheox, or stearalkonium hectorite, which is obtainable, for example, under the trade name Softisan® Gel from Hüls AG).

Preparations according to the invention can advantageously also comprise oil thickeners in order to improve the tactile properties of the emulsion and the stick consistency. Advantageous oil thickeners for the purposes of the present invention are, for example, other solids, such as, for example, hydrophobic silicon oxides of the Aerosil® type, which are obtainable from Degussa AG. Advantageous Aerosil® products are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and/or Aerosil® R976.

In addition, so-called metal soaps (i.e. the salts of higher fatty acids with the exception of the alkali metal salts) are also advantageous oil thickeners for the purposes of the present invention, such as, for example, aluminum stearate, zinc stearate and/or magnesium stearate.

It is likewise advantageous to add amphoteric or zwitterionic surfactants (e.g. cocoamidopropylbetaine) and moisturizers (e.g. betaine) to preparations according to the invention. Examples of amphoteric surfactants which are to be used advantageously are acyl-/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of interface- or surface-active substances (one or more compounds) in the preparations according to the invention is preferably from 0.001 to 30% by weight, particularly preferably 0.05-20% by weight, in particular 1-10% by weight, based on the total weight of the preparation.

A surprising property of the preparations according to the invention is that they are very good vehicles for cosmetic or dermatological active ingredients into the skin, preferred active ingredients being the antioxidants mentioned above which are able to protect the skin against oxidative stress.

The active ingredients (one or more compounds) can also very advantageously be chosen according to the invention from the group of lipophilic active ingredients, in particular from the following group:
acetylsalicylic acid, atropine, azulene, hydrocortisone and derivatives thereof, e.g. hydrocortisone-17 valerate, vitamins of the B and D series, very favorably vitamin B₁, vitamin B₁₂ and vitamin D₁, but also bisabolol, unsaturated fatty acids, namely the essential fatty acids (often also called vitamin F), in particular gamma-linolenic acid, oleic acid, eicosapentaenoic acid, docosahexaenoic acid and derivatives thereof, chloroamphenicol, caffeine, prostaglandins, thymol, camphor, extracts or other products of vegetable and animal origin, e.g. evening primrose oil, borage oil or currant seed oil, fish oils, cod-liver oil and also ceramides and ceramide-like compounds, etc.

It is also advantageous to choose the active ingredients from the group of refatting substances, for example purcellin oil, Eucerit® and Neoceri®.

The active ingredient(s) is/are also particularly advantageously chosen from the group of NO synthase inhibitors, particularly if the preparations according to the invention are to be used for the treatment and prophylaxis of the symptoms of intrinsic and/or extrinsic skin aging and for the treatment and prophylaxis of the harmful effects of ultraviolet radiation on the skin.

A preferred NO synthase inhibitor is nitroarginine.

The active ingredient(s) is/are also advantageously chosen from the group which includes catechins and bile esters of catechins and aqueous or organic extracts from plants or parts of plants which have a content of catechins or bile esters of catechins, such as, for example, the leaves of the Theaceae plant family, in particular of the species Camellia sinensis (green tea). Particularly advantageous are typical ingredients thereof (such as e.g. polyphenols or catechins, caffeine, vitamins, sugar, minerals, amino acids, lipids).

Catechins are a group of compounds which are to be regarded as hydrogenated flavones or anthocyanidines and are derivatives of “catechin” (catechol, 3,3′,4′,5,7-flavanpentol, 2-(3,4-dihydroxyphenyl)chroman-3,5,7-triol). Epicatechin ((2R,3R)-3,3′,4′,5,7-flavanpentol) is also an advantageous active ingredient for the purposes of the present invention.

Also advantageous are plant extracts with a content of catechins, in particular extracts of green tea, such as e.g. extracts from leaves of plants of the species Camellia spec., very particularly the types of tea Camellia sinenis, C. assamica, C. taliensis and C. irrawadiensis and hybrids of these with, for example, Camellia japonica.

Preferred active ingredients are also polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin and (−)-epigallocatechin gallate.

Flavone and its derivatives (also often collectively called “flavones”) are also advantageous active ingredients for the purposes of the present invention. They are characterized by the following basic structure (substitution positions are shown):

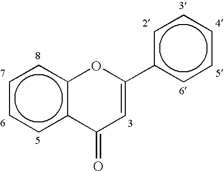

Some of the more important flavones, which can also preferably be used in preparations according to the invention, are given in the Table 2 below:

TABLE 2
OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula 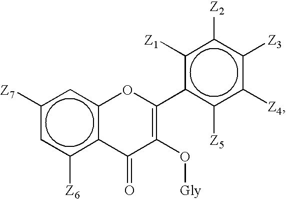
where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can however, also advantageously be chosen from the group of substances of the generic structural formula 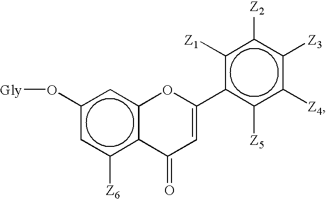
where Z₁ to Z₆, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

Preferably, such structures can be chosen from the group of substances of the generic structural formula 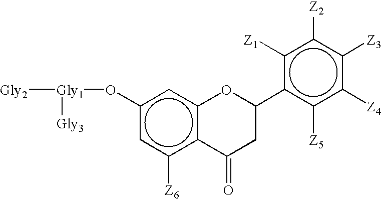
where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

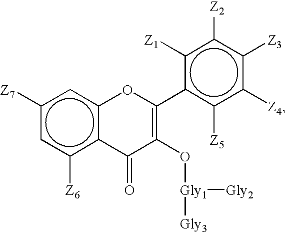

The flavone glycosides according to the invention are particularly advantageously chosen from the group given by the following structure: 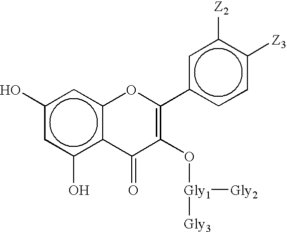
where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glycoside(s) from the group consisting of α-glucosylrutin, α-glucosylmyricetin, α-glucosylisoquercitrin, α-glucosylisoquercetin and α-glucosylquercitrin.

Particular preference is given, according to the invention, to α-glucosylrutin.

Also advantageous according to the invention are naringin (aurantin, naringenin-7-rhamnoglucoside), hesperidin (3′,5,7-trihydroxy-4′-methoxyflavanone-7-rutinoside, hesperidoside, hesperetin-7-O-rutinoside). Rutin (3,3′,4′,5,7-pentahydroxyflyvone-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6 deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol-7-glucoside (3′,4′,5,7-tetrahydroxyflavanone-7 glucoside), flavanomarein (3′,4′,7,8-tetrahydroxyflavanone-7 glucoside) and isoquercetin (3,3′,4′,5,7-pentahydroxyflavanone-3-(β-D-glucopyranoside). It is also advantageous to choose the active ingredient(s) from the group of ubiquinones and plastoquinones.

Ubiquinones are characterized by the structural formula 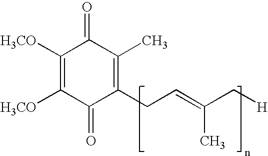
and are the most widespread and thus the most investigated bioquinones. Ubiquinones are referred to depending on the number of isoprene units linked in the side chain as Q-1, Q-2, Q-3 etc., or depending on the number of carbon atoms, as U-5, U-10, U-15 etc. They preferably appear with certain chain lengths, e.g. in some microorganisms and yeasts where n=6. In most mammals including man, Q10 predominates.

Coenzyme Q10 is particularly advantageous and is characterized by the following structural formula:

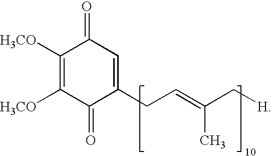

Plastoquinones have the general structural formula

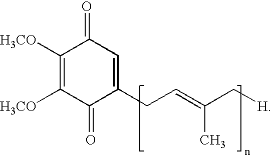

Plastoquinones differ in the number n of isoprene radicals and are referred to accordingly, e.g. PQ-9 (n=9). In addition, other plastoquinones with varying substituents on the quinone ring exist.

Creatine and/or creatine derivatives are preferred active ingredients for the purposes of the present invention. Creatine is characterized by the following structure:

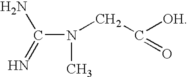

Preferred derivatives are creatine phosphate and creatine sulphate, creatine acetate, creatine ascorbate and the derivatives esterified at the carboxyl group with mono- or polyfunctional alcohols.

A further advantageous active ingredient is L-carnitine [3-hydroxy-4-(trimethylammonio)-butyrobetaine]. Acylcarnitines chosen from the group of substances of the following general structural formula 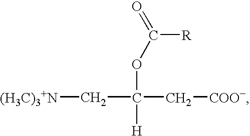
where R is chosen from the group of branched and unbranched alkyl radicals having up to 10 carbon atoms, are also advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form.

Further advantageous active ingredients are sericoside, pyridoxol, vitamin K, biotin and aroma substances.

The list of said active ingredients and active ingredient combinations which can be used in the preparations according to the invention is, of course, not intended to be limiting. The active ingredients can be used individually or in any combinations with one another.

The amount of such active ingredients (one or more compounds) in the preparations accord-ing to the invention is preferably 0.001 to 30% by weight, particularly preferably 0.05-20% by weight, in particular 1-10% by weight, based on the total weight of the preparation.

The preparations according to the invention are produced under the conditions known to the person skilled in the art. Usually, the constituents of the oil phase and of the water phase are combined separately and heated and then combined with stirring and, particularly advantageously, with homogenization, very particularly advantageously with stirring with moderate to high energy input, advantageously using a toothed-wheel dispersing machine with a speed of at most 10 000 rpm, preferably from 2500 to 7700 rpm.

The examples below are intended to illustrate the present invention.

### EXAMPLE 1

### O/W Cream

| | % by weight |
|---|---|
| Stearic acid | 2.00 |
| Lignoceric acid | 1.00 |
| Cetyl alcohol | 5.00 |
| PEG-20 stearate | 1.00 |
| Sorbitan stearate | 1.00 |
| Caprylic/capric triglycerides | 3.00 |
| Glyceryl stearate | 1.00 |
| Octyldodecanol | 6.00 |
| Isopropyl palmitate | 1.00 |
| Octyl stearate | 1.00 |
| Cyclomethicone | 3.00 |
| Myristyl myristate | 3.00 |
| Glycerol | 3.00 |
| Carbomer | 0.10 |
| Ceramide III | 0.50 |
| Sodium hydroxide | q.s. |
| Preservative | q.s. |
| Perfume | q.s. |
| Water, demineralized | ad 100.00 |
| pH adjusted to 5.0-6.5 | |

The constituents of the oil phase and of the water phase are combined separately and heated, and then combined together with homogenization, using a toothed-wheel dispersing machine at a speed of 5000 rpm.

### EXAMPLE 2

### O/W Lotion

| | % by weight |
|---|---|
| Cholesterol | 2.50 |
| Lanosterol | 0.50 |
| Lanolin | 0.50 |
| Myristyl alcohol | 1.00 |
| Cetylstearyl alcohol | 2.00 |
| PEG-100 stearate | 1.00 |
| Sorbitan stearate | 1.00 |
| Glyceryl stearate | 1.00 |
| Propylene glycol dicaprylate/dicaprate | 3.00 |
| Octyl palmitate | 5.00 |
| Hydrogenated polyisobutene | 2.00 |
| Glycerol | 3.00 |
| Carbomer | 0.15 |
| Alpha-glucosylrutin | 0.50 |
| Retinol | 0.20 |
| Potassium hydroxide | q.s. |
| Preservative | q.s. |
| Perfume | q.s. |
| Water, demineralized | ad 100.00 |
| pH adjusted to 5.0 | |

The constituents of the oil phase and of the water phase are combined separately and heated, and then combined together with homogenization, using a toothed-wheel dispersing machine at a speed of 7000 rpm.

### EXAMPLE 3

### O/W Lotion

| | % by weight |
|---|---|
| Stearic acid | 3.00 |
| Myristyl alcohol | 1.00 |
| Cetylstearyl alcohol | 1.00 |
| PEG-100 stearate | 1.00 |
| Sorbitan stearate | 1.00 |
| Glyceryl stearate | 1.00 |
| Propylene glycol dicaprylate/dicaprate | 3.00 |
| Paraffin oil | 5.00 |
| Glycerol | 3.00 |
| Carbomer | 0.15 |
| Alpha-glucosylrutin | 0.50 |
| Retinol | 0.20 |
| Potassium hydroxide | q.s. |
| Preservative | q.s. |
| Perfume | q.s. |
| Water, demineralized | ad 100.00 |
| pH adjusted to 5.0 | |

The constituents of the oil phase and of the water phase are combined separately and heated, and then combined together with homogenization, using a toothed-wheel dispersing machine at a speed of 3200 rpm.

### EXAMPLE 4

### O/W Cream

| | % by weight |
|---|---|
| PEG-40 stearate | 0.50 |
| Stearic acid | 3.00 |
| Cholesterol | 1.00 |
| Cetylstearyl alcohol | 4.00 |
| Glyceryl stearate | 1.00 |
| Sorbitan stearate | 0.50 |
| Caprylic/capric triglycerides | 3.00 |
| Dimethicone | 4.00 |
| Dicaprylyl ether | 2.00 |
| Hydrogenated cocnut fatty acid glycerides | 5.00 |
| Ceramide II | 0.07 |
| Xanthan gum | 0.10 |
| Citric acid | 0.10 |
| Glycerol | 3.00 |
| Perfume, preservative, dyes etc. | q.s. |
| Water | ad 100.00 |
| pH adjusted to 5.0-7.0 | |

The constituents of the oil phase and of the water phase are combined separately and heated, and then combined together with homogenization, using a toothed-wheel dispersing machine at a speed of 5000 rpm.

### EXAMPLE 5

### O/W Emulsion Make-Up

| | % by weight |
|---|---|
| PEG-40 stearate | 1.00 |
| Stearic acid | 3.00 |
| Lanolin | 0.50 |
| Cholesterol | 0.25 |
| Lignoceric acid | 0.25 |
| Cetyl alcohol | 2.00 |
| Glyceryl stearate | 1.00 |
| Sorbitan stearate | 1.00 |
| Caprylic/capric triglycerides | 3.00 |
| Cetylstearyl isononanoate | 2.00 |
| Hexyldecanol | 5.00 |
| Glycerol | 3.00 |
| Carbomer | 0.15 |
| Mica | 1.00 |
| Magnesium silicate | 1.00 |
| Iron oxide | 1.00 |
| Titanium dioxide | 2.50 |
| Talc | 5.00 |
| Retinol | 0.20 |
| Sodium hydroxide | q.s. |
| Preservative | q.s. |
| Perfume | q.s. |
| Water, demineralized | ad 100.00 |
| pH adjusted to 6.5 | |

The constituents of the oil phase and of the water phase are combined separately and heated, and then combined together with homogenization, using a toothed-wheel dispersing machine at a speed of 6000 rpm.

### EXAMPLE 6

### O/W Cream

| | % by weight |
|---|---|
| PEG-100 stearate | 1.00 |
| Lignoceric acid | 1.00 |
| Cholesterol | 1.00 |
| Lanolin | 1.00 |
| Cetyl alcohol | 4.00 |
| Sorbitan stearate | 1.00 |
| Glyceryl distearate | 1.00 |
| Octyldodecyl myristate | 3.00 |
| Octyldodecanol | 3.00 |
| Paraffin oil | 2.00 |
| Butylene glycol caprylate/caprate | 2.00 |
| Dimethicone | 2.00 |
| Cyclomethicone | 1.00 |
| Ceramide | 0.50 |
| Vitamin E acetate | 1.00 |
| Xanthan gum | 0.10 |
| Glycerol | 3.00 |
| BHT | 0.02 |
| Disodium EDTA | 0.10 |
| Perfume, preservative, dyes | q.s. |
| Water | ad 100.00 |
| pH adjusted to 5.0-6.5 | |

The constituents of the oil phase and of the water phase are combined separately and heated, and then combined together with homogenization, using a toothed-wheel dispersing machine at a speed of 2800 rpm.

### EXAMPLE 7

### O/W Lotion

| | % by weight |
|---|---|
| PEG-100 stearate | 0.50 |
| Stearic acid | 2.00 |
| Cetyl alcohol | 2.00 |
| Sorbitan stearate | 0.50 |
| Glyceryl stearate | 0.50 |
| C₁₂₋₁₅ Alkylbenzoate | 4.00 |
| Caprylic/capric triglyceride | 3.00 |
| Paraffin oil | 2.00 |
| Tocopheryl acetate | 2.00 |
| Sodium carbomer | 0.10 |
| Ceramide III | 0.50 |
| Glycerol | 3.00 |
| Perfume, preservative, dyes etc. | q.s. |
| Water | ad 100.00 |
| pH adjusted to 5.0-7.0 | |

The constituents of the oil phase and of the water phase are combined separately and heated, and then combined together with homogenization, using a toothed-wheel dispersing machine at a speed of 3000 rpm.

### EXAMPLE 8

### O/W Cream

| | % by weight |
|---|---|
| PEG-100 stearate | 0.50 |
| Stearic acid | 3.00 |
| Cetyl alcohol | 5.00 |
| Sorbitan stearate | 0.50 |
| Glyceryl stearate | 0.50 |
| Dimethicone | 1.00 |
| Paraffin oil | 3.00 |
| Myristyl myristate | 2.00 |
| Hydrogenated coconut fatty acid glyceride | 4.00 |
| Tocopheryl acetate | 2.00 |
| Sodium carbomer | 0.10 |
| Ceramide III | 0.50 |
| Glycerol | 3.00 |
| Perfume, preservative, dyes, etc. | q.s. |
| Water | ad 100.00 |
| pH adjusted to 5.0-7.0 | |

The constituents of the oil phase and of the water phase are combined separately and heated, and then combined together with homogenization, using a toothed-wheel dispersing machine at a speed of 5200 rpm.

### EXAMPLE 9

### Sunscreen Cream

| | % by weight |
|---|---|
| PEG-100 stearate | 1.00 |
| Stearic acid | 2.00 |
| Cholesterol | 1.00 |
| Lignoceric acid | 1.00 |
| Cetyl alcohol | 4.00 |
| Sorbitan stearate | 1.00 |
| Glyceryl stearate | 1.50 |
| Octyldodecanol | 2.00 |
| Paraffin oil | 6.00 |
| Butyloctanol | 1.00 |
| Xanthan gum | 0.10 |
| Sodium carbomer | 0.10 |
| Glycerol | 3.00 |
| Ceramide III | 0.20 |
| BHT | 0.02 |
| Na₂H₂EDTA | 0.10 |
| Perfume, preservative, dyes, etc. | q.s. |
| Water | ad 100.00 |
| pH adjusted to 5.0-5.5 | |

The constituents of the oil phase and of the water phase are combined separately and heated, and then combined together with homogenization, using a toothed-wheel dispersing machine at a speed of 3500 rpm.

1-7. (canceled)

8. A cosmetic or dermatological composition, wherein the composition comprises:
(I) from 1% to 5% by weight of at least one substance which is selected from sterols and C₁₂-C₄₀-fatty acids;
(II) from 0.1% to 1.5% by weight of at least one ester of one or more C₁₄-C₄₀-fatty acids with one or more of glycerol, propylene glycol and glycol;
(III) from 0.1% to 1.5% by weight of at least one ester of a C₁₄-C₄₀-fatty acid of sorbitan, which ester may be ethoxylated with a degree of ethoxylation of up to 100;
(IV) from 0.1% to 1.5% by weight of at least one ethoxylated C₁₂-C₄₀-fatty acid having a degree of ethoxylation of up to 100; and
(V) from 0.5% to 7% by weight of at least one C₁₂-C₄₀-fatty alcohol.

9. The composition of claim 8, wherein the composition comprises at least one sterol.

10. The composition of claim 9, wherein the at least one sterol comprises at least one of cholesterol and lanosterol.

11. The composition of claim 10, wherein the composition comprises cholesterol.

12. The composition of claim 8, wherein the composition comprises at least one C₁₂-C₄₀-fatty acid.

13. The composition of claim 12, wherein the at least one C₁₂-C₄₀-fatty acid comprises at least one of stearic acid, palmitic acid and lignoceric acid.

14. The composition of claim 13, wherein the composition comprises stearic acid.

15. The composition of claim 8, wherein the composition comprises at least one C₁₂-C₄₀-fatty acid and at least one sterol.

16. The composition of claim 15, wherein the composition comprises cholesterol and stearic acid.

17. The composition of claim 8, wherein the composition comprises at least one of glycerol monostearate, glycerol distearate, propylene glycol monostearate, glycerol isostearate, glycerol lanolate, glycerol myristate, glycerol laurate, glycerol oleate and glycerol stearate citrate.

18. The composition of claim 17, wherein the composition comprises at least one of glycerol monostearate and glycerol distearate.

19. The composition of claim 8, wherein the composition comprises glycerol monostearate.

20. The composition of claim 8, wherein the composition comprises at least one of sorbitan stearate, sorbitan distearate, sorbitan isostearate, sorbitan oleate, PEG-40 sorbitan peroleate, PEG-40 sorbitan perisostearate and sorbitan sesquioleate.

21. The composition of claim 20, wherein the composition comprises sorbitan stearate.

22. The composition of claim 8, wherein the at least one ester of a C₁₄-C₄₀-fatty acid of sorbitan comprises an ester which has a degree of ethoxylation of at least 10.

23. The composition of claim 8, wherein at least one ethoxylated C₁₂-C₄₀-fatty acid having a degree of ethoxylation of up to 100 comprises at least one ester which has a degree of ethoxylation of at least 5.

24. The composition of claim 20, wherein the composition comprises at least one of PEG-20 stearate, PEG-30 stearate, PEG-40 stearate, and PEG-100 stearate.

25. The composition of claim 19, wherein the composition comprises PEG-100 stearate.

26. The composition of claim 8, wherein the at least one C₁₂-C₄₀-fatty alcohol comprises at least one of myristyl alcohol, cetyl alcohol, isocetyl alcohol, cetylstearyl alcohol, stearyl alcohol, isostearyl alcohol and behenyl alcohol.

27. The composition of claim 8, wherein the at least one C₁₂-C₄₀-fatty alcohol comprises at least one wool wax alcohol.

28. The composition of claim 8, wherein a weight ratio (II): (III): (IV) is a b c and a, b and c independently are rational numbers of from 1 to 5.

29. The composition of claim 28, wherein a, b and c independently are rational numbers of from 1 to 3.

30. The composition of claim 28, wherein a weight ratio [(II)+(III)+(IV)]: (V) is from 5:1 to 1:5.

31. The composition of claim 30, wherein the composition has a pH of from 3.5 to 8.

32. The composition of claim 8, wherein the composition has a pH of from 4.5 to 6.5.

33. The composition of claim 8, wherein components (I) to (V) are present in a total amount of at least about 5.5% by weight.

34. The composition of claim 33, wherein components (I) to (V) are present in a total amount of not more than about 11.5% by weight.

35. The composition of claim 32, wherein the composition further comprises at least 2% by weight of liquid lipids.

36. The composition of claim 35, wherein the liquid lipids comprise at least one of a Guerbet alcohol, a saturated triglyceride, an ether of a medium-chain fatty alcohol, a nonpolar lipid, a silicone oil and a dialkyl carbonate.

37. A cosmetic or dermatological composition, wherein the composition comprises:
(I) from 1% to 5% by weight of at least one substance which is selected from sterols and C₁₂-C₄₀-fatty acids, which substance comprises at least one of cholesterol, lanosterol, stearic acid, palmitic acid and lignoceric acid;
(II) from 0.1% to 1.5% by weight of at least one ester of one or more C₁₄-C₄₀-fatty acids with one or more of glycerol, propylene glycol and glycol, which ester comprises at least one of glycerol monostearate, glycerol distearate, propylene glycol monostearate, glycerol isostearate, glycerol lanolate, glycerol myristate, glycerol laurate, glycerol oleate and glycerol stearate citrate;
(III) from 0.1% to 1.5% by weight of at least one ester of a C₁₄-C₄₀-fatty acid of sorbitan, which ester may be ethoxylated with a degree of ethoxylation of up to 100 and comprises at least one of sorbitan stearate, sorbitan distearate, sorbitan isostearate, sorbitan oleate, PEG-40 sorbitan peroleate, PEG-40 sorbitan perisostearate and sorbitan sesquioleate;
(IV) from 0.1% to 1.5% by weight of at least one ethoxylated C₁₂-C₄₀-fatty acid having a degree of ethoxylation of up to 100, which ethoxylated fatty acid comprises at least one of PEG-20 stearate, PEG-30 stearate, PEG-40 stearate and PEG-100 stearate; and
(V) from 0.5% to 7% by weight of at least one C₁₂-C₄₀-fatty alcohol, which fatty alcohol comprises at least one of myristyl alcohol, cetyl alcohol, isocetyl alcohol, cetylstearyl alcohol, stearyl alcohol, isostearyl alcohol and behenyl alcohol myristyl alcohol, cetyl alcohol, isocetyl alcohol, cetylstearyl alcohol, stearyl alcohol, isostearyl alcohol and behenyl alcohol.

38. The composition of claim 37, wherein the composition comprises:
(I) from 1% to 5% by weight of at least one of cholesterol, lanosterol, stearic acid, palmitic acid and lignoceric acid;
(II) from 0.1% to 1.5% by weight of at least one of glycerol monostearate, glycerol distearate, propylene glycol monostearate, glycerol isostearate, glycerol lanolate, glycerol myristate, glycerol laurate, glycerol oleate and glycerol stearate citrate;
(III) from 0.1% to 1.5% by weight of at least one of sorbitan stearate, sorbitan distearate, sorbitan isostearate, sorbitan oleate, PEG-40 sorbitan peroleate, PEG-40 sorbitan perisostearate and sorbitan sesquioleate;
(IV) from 0.1% to 1.5% by weight of at least one of PEG-20 stearate, PEG-30 stearate, PEG-40 stearate and PEG-100 stearate; and
(V) from 0.5% to 7% by weight of at least one of myristyl alcohol, cetyl alcohol, isocetyl alcohol, cetylstearyl alcohol, stearyl alcohol, isostearyl alcohol and behenyl alcohol.

39. A cosmetic or dermatological composition, wherein the composition comprises:
(I) from 1% to 5% by weight of at least one substance which is selected from sterols and C₁₂-C₄₀-fatty acids;
(II) from 0.1% to 1.5% by weight of at least one ester of one or more even-numbered C₁₂-C₄₀-fatty acids with one or more of glycerol and a glycol;
(III) from 0.1% to 1.5% by weight of at least one ester of a C₁₂-C₄₀-fatty acid of a sorbitan, which ester may be ethoxylated with a degree of ethoxylation of from 10 to 100;
(IV) from 0.1% to 1.5% by weight of at least one ethoxylated C₁₂-C₄₀-fatty acid having a degree of ethoxylation of from 5 to 100; and
(V) from 0.5% to 7% by weight of at least one C₁₂-C₄₀-fatty alcohol.

40. A skin lotion which comprises the composition of claim 8.

41. A skin protection cream which comprises the composition of claim 8.

42. A cosmetic milk which comprises the composition of claim 37.

43. A sunscreen lotion which comprises the composition of claim 8.

44. The composition of claim 8, wherein the composition further comprises at least one UV absorbing substance.

45. The composition of claim 8, wherein the composition further comprises at least one antioxidant.

46. The composition of claim 8, wherein the composition further comprises at least one of a cosmetically active ingredient and a dermatologically active ingredient.

47. A method for the cosmetic or dermatological treatment of skin, wherein the method comprises applying onto at least parts of the skin the composition of claim 8.
